(19) United States
(12) Patent Application Publication
(10) Pub. No.: US 2016/0199298 A1
(43) Pub. Date: Jul. 14, 2016

(54) FOSFESTROL FOR USE IN CURATIVE OR PALLIATIVE TREATMENT OF CANCER IN FEMALE MAMMALS

(71) Applicant: CHAMAELEO PHARMA BVBA, Diegem (BE)

(72) Inventors: Johannes Jan PLATTEEUW, Diegem (BE); Martijn DE BREE, Almere (NL); Edwin KLUMPER, Amstelveen (NL)

(73) Assignee: CHAMAELEO PHARMA BVBA, Diegem (BE)

(21) Appl. No.: 14/435,732

(22) PCT Filed: Oct. 14, 2013
(86) PCT No.: PCT/EP2013/071431

§ 371 Date: Apr. 14, 2015

(30) Foreign Application Priority Data
Oct. 15, 2012 (EP) 12188524.8

(51) Int. Cl. A61K 9/00; A61K 9/20; A61K 31/6615

(52) U.S. Cl. CPC A61K 9/0053; A61K 31/6615; A61K 9/20

(57) ABSTRACT

The present invention concerns the use of Fosfetrol (diethylstilbestrol diphosphate) in a method of curative or palliative treatment of cancer in female mammals, said method comprising orally administering Fosfestrol in a daily dosage of at least 500 mg. Examples of cancers that can be treated by the present method include breast cancer, endometrium cancer and ovarian cancer. The invention further provides an oral dosage unit containing at least 500 mg of Fosfestrol.

### TECHNICAL FIELD OF THE INVENTION

The present invention relates to the use of Fosfestrol in curative or palliative treatment of cancer in a female mammal, said treatment comprising oral administration of the Fosfestrol in a daily amount of at least 500 mg. Examples of cancers that can be treated by the present method include breast cancer, endometrium cancer and ovarian cancer.

The invention also provides an oral dosage unit containing at least 500 mg of Fosfestrol.

### BACKGROUND OF THE INVENTION

Cancer is still among the major causes of death in the western world. This applies to both males and females. Breast cancer is the most frequently occurring cancer in females. Due to ongoing research on new medicines and methods of treatment, life expectance of people suffering from different types of cancer has steadily increased over the years. Nevertheless, better medicines and enhanced methods of treatment are still needed.

Endocrine treatment essentially adds, blocks, or removes hormones. To slow or stop the growth of certain cancers (such as breast cancer), synthetic hormones or other drugs may be given to block the body's natural hormones. Sometimes surgery is needed to remove the gland that makes a certain hormone. Endocrine therapy is also known as hormonal therapy, hormone therapy and hormone treatment.

### DES Therapy

Among the medicines that already have been used in the treatment of cancer is diethylstilbestrol (DES). DES is a synthetic nonsteroidal estrogen that was first synthesized in 1938. It was designed to achieve castrate levels of testosterone. Testosterone drives prostate cancer growth and withdrawal of testosterone by surgical castration was the first hormone ablation therapy in prostate cancer treatment. DES was developed to achieve chemical castration by inhibiting testicular production of testosterone.

However, the role of oral administration of DES in the treatment of prostate cancer has been limited because of an association with thromboembolic toxicity. When estrogens like for example DES are given orally, they are subject to the intestinal and hepatic first-pass effect leading to high hormone concentrations in the liver promoting the synthesis of clotting proteins like fibrinogen.

Non-cancer related deaths, mostly cardiovascular in origin, were increased by 36% in patients suffering from prostate cancer receiving 5 mg of DES p.o. per day (Byar D P: Proceedings: The Veterans Administration Cooperative Urological Research Group's studies of cancer of the prostate. Cancer (1973) 32:1126-30). Other studies evaluating lower doses of DES reported similar efficacy towards testosterone suppression as obtained with the 5 mg dose and acceptable thromboembolic toxicity. This led to the adoption of 3 mg per day as the most commonly used DES oral dose for treating prostate cancer. However, the thromboembolic toxicity remained a concern.

The nail in the coffin for DES as a first line therapy in prostate cancer came when a study was published in 1984 by the Leuprolide Study Group comparing the efficacy and safety of 3 mg DES versus Leuprolide in metastatic prostate cancer, which showed similar therapeutic efficacy but a much improved safety profile for Leuprolide (The Leuprolide Study Group (1984) Leuprolide versus diethylstilbestrol for metastatic prostate cancer. N Engl J Med ; 311(20):1281-6).

### DES Therapy in Breast Cancer:

High-dose DES therapy also was the endocrine treatment of choice in postmenopausal women with advanced breast cancer prior to the introduction of tamoxifen in the 1970s. Cole et al (A new anti-oestrogenic agent in late breast cancer. An early clinical appraisal of IC14647 4.Br J Cancer 1971, 25:270-275) reported the first clinical trial of tamoxifen in women with late or recurrent breast cancer, and compared their findings with those from another similarly conducted trial in which women received diethylstilbestrol (DES) or an androgen. They concluded that the level of response was of the same order for the three agents but that an advantage for tamoxifen was the low incidence of ‘troublesome side effects’.

Consistent with these early findings, the acceptance of tamoxifen as preferable to estrogen therapy was based not on a superior efficacy, but rather on an improved tolerability demonstrated in phase III trials (Ingle et al. Randomized clinical trial of diethylstilbestrol versus tamoxifen in postmenopausal women with advanced breast cancer. N Engl J Med 1981, 304:16-21.)

### Re-Appraisal of DES in Breast Cancer

A re-appraisal of DES as a treatment option for breast cancer came when it became clear from long term survival studies in e.g. breast cancer that there was no significant difference between DES and tamoxifen in terms of response rates and time to progression. However, survival was modestly and significantly better for women initially treated with DES (adjusted P=0.039), with median survivals of 3.0 years versus 2.4 years, and 5-year survivals of 35% and 16%, respectively (Peethambaram et al. Randomized trial of diethylstilbestrol vs. tamoxifen in postmenopausal women with metastatic breast cancer. An updated analysis. Breast Cancer Res Treat, 5 (1999)4:117-122)

A prospective phase II study by Lonning et al, (High-dose estrogen treatment in postmenopausal breast cancer patients heavily exposed to endocrine therapy. Breast Cancer Res Treat, 67 (2001):111-116) further showed that 15 mg DES/daily (p.o.) is effective in highly pretreated patients. The fact that high-dose estrogens are not tolerable in all patients is illustrated by the fact that six patients (19%) stopped therapy because of side effects.

### Fosfestrol Therapy

GB 732,286 describes the synthesis of Fosfestrol (diethylstilbestrol diphosphate). Fosfestrol was developed as a prodrug of DES to achieve safe inhibition of testosterone production without causing thromboembolic side effects caused by free DES. The phosphate groups were added to inactivate DES, thereby circumventing the intestinal and hepatic first pass effect and decreasing the circulating levels of free DES. Fosfestrol itself was considered to be inactive and it was known that prostate cancer cells have increased expression of prostate acid phosphatase (PAP). It was thought that PAP would remove the phosphate groups and release DES near its side of action.

Fosfestrol was introduced and marketed in the 1950's under the name Honvan® and has been successfully applied in the treatment of prostate cancer for many years. Since fosfestrol is a prodrug of DES and since high plasma levels of DES have been associated with thromboembolic side effects, there has been a reluctance to orally administer fosfestrol in high dosages, notably dosages in excess of 360 mg p.o. per day.

With a view to its estrogenic effect on testosterone decrease an oral dose of 200 mg of fosfestrol is deemed to be equipotent to an oral dose of 3 mg DES. Based on the increase of prolactin level in the blood, a daily oral dosage of 360 mg fosfestrol or a daily intravenous dosage of 1200 mg fosfestrol are regarded to be equipotent to a daily oral dosage of 1 mg DES. Clinical results than showed that fosfestrol is safe and effective to use in treating hormone-dependent prostate cancer at oral doses of 200-360 mg divided in two or three dosages per day.

Oelschläger et al. (New Results on the Pharmacokinetics of Fosfestrol, Urol. Int. 43 (1988), 15-21) have shown that Fosfestrol and its monophosphate exist only for a short time in small amounts in the circulating blood after intravenous administration (1.5 g per day for 10 days), whilst after oral administration (360 mg), not even traces of the phosphates could be detected in the plasma

Schneider et al. (Effects of diethylstilbestrol and its mono and diphosphate on experimental mammary tumors and prostatic tumors, Urol Int. 43, (1988) 10-14) also tested the mammary tumor inhibiting activity of DES, DES diphosphate and DES mono phosphate in the MCF-7 cells in vitro. Cells were incubated for 4 days with the test compounds at concentrations of 1, 5 and 10 μM. At the lowest concentration no compound exerted any growth inhibition. At 5 μM (2 μg/ml) the diphosphate showed a 50% reduction in cell growth, whilst DES and DES monophosphate showed no effect at all. At the highest concentration all compounds almost completely inhibited the growth of tumor cells. The authors concluded that a cytotoxic effect of DES diphosphate and of DES monophosphate and DES itself was shown albeit at relatively high concentrations.

In the same publication, Schneider (Discussion of the experimental papers. Urol Int 43 (1988), 23, also states “I think there is no general cytotoxic effect of DES on hormone independent tumors. The point is that we have found cytotoxic or direct effect on prostatic tumors, in tumors which are also hormone responsive. We have used DES in other models that I have not shown, e.g. hormone independent mammary carcinomas with very high doses in vivo and there was no effect.”.

### Fosfestrol Therapy in the Treatment of Breast Cancer

Fosfestrol has also been used in the treatment of breast cancer. Rosset et al. Palliative treatment of metastatic breast cancer with diethyldioxystilbene diphosphate (Honvan), Schweiz Med Wochenschr., (1975) October 25;105(43):1388-90 performed a small study in which a daily dose of 100-120 mg fosfestrol was administered orally to 21 metastatic breast cancer patients. Of these patients 33% had an objective remission, 38.5% remained unchanged and 28.5% were progressive. In half of the progressive patients tumor growth actually accelerated.

### SUMMARY OF THE INVENTION

The present inventors have found that Fosfestrol (diethylstilbestrol diphosphate), if administered orally in a high daily amount of at least 500 mg, can effectively be used in the treatment of cancer in female mammals, especially in the treatment of a cancer selected from breast cancer, endometrium cancer and ovarian cancer. The present method is particularly suited for treating hormone insensitive sub-types of these intitially hormone senstive cancers.

Unexpectedly, the inventors have discovered that Fosfestrol, when administered in such high dosages does not give rise to serious side effects, such as thromboembolic toxicity or even mortality.

Without wishing to be bound by theory it is hypothesized that the toxic effects that have been observed in the past for orally administered DES (˜3 mg daily) are not so much caused by DES itself, but by oxidized DES metabolites.

When DES is given orally it is subjected to intensive intestinal and hepatic metabolism. Metabolisation of orally administered DES occurs through oxidation followed by conjugation or through direct conjugation. The main oxidative reactions are hydroxylation of the aromatic rings and, at the ethyl group, subsequent conjugations. Also formation of a hexadiene has been observed. The conjugates formed are sulphates, or glucuronides or combinations of the two.

The inventors believe that orally administered Fosfestrol is less prone to oxidation than orally administered DES. Thus, higher DES plasma levels can be achieved with orally administered Fosfestrol compared to orally administered DES, but with substantially lower levels of oxidized DES metabolites and consequently with significantly less toxic side-effects.

The present invention therefore provides a way to achieve plasma levels of free DES above 1 microgram/ml with limited side-effects by administering a high oral dose of Fosfestrol.

The present invention also relates to an oral dosage unit that contains at least 500 mg of Fosfestrol.

### DETAILED DESCRIPTION OF THE INVENTION

A first aspect of the invention concerns Fosfestrol (diethylstilbestrol diphosphate) for use in a method of curative or palliative treatment of cancer in female mammals, said method comprising orally administering Fosfestrol in a daily dosage of at least 500 mg.

The term ‘Fosfestrol’ as used herein refers to a diethylstilbestrol moiety of which both the hydroxyl groups are phosphated. The term ‘Fosfestrol’ encompasses pharmaceutically acceptable salts of Fosfestrol.

The term ‘pharmaceutically acceptable salt’, as used herein, means those salts of compounds of the invention that are safe and effective for use in mammals and that possess the desired biological activity. Descriptions of counter ions for pharmaceutically acceptable salts of pharmaceutical compounds can be found in P. Heinrich Stahl, Camille G. Wermuth (Eds.), Handbook of Pharmaceutical Salts, Properties, Selection and Use, Wiley VCH (2002).

The diethylstilbestrol moiety in the DES phosphate of the present invention may be in the trans-form or the cis-form. Naturally, also mixtures of the trans- and cis-form may be employed.

The term ‘cancer’ as used herein refers to a malignant neoplasm involving unregulated cell growth. In cancer, cells divide and grow uncontrollably, forming malignant tumors, and invade nearby parts of the body.

The term ‘curative treatment’ as used herein refers to a treatment that aims to cure a disease or to improve symptoms associated with a disease.

The term ‘palliative treatment’ as used herein refers to a treatment or therapy that does not aim at curing a disease but rather at providing relief

The term ‘oral’ as used herein, unless indicated otherwise, is synonymous to ‘per oral’.

The term ‘dosage’ as used herein refers to the amount of a pharmaceutically active substance that is administered to a mammal. Hence, the term ‘dosage’ does not include any carrier or other pharmaceutically acceptable excipient that is part of a ‘dosage unit’ to be administered.

In this document and in its claims, the verb ‘to comprise’ and its conjugations are used in their non-limiting sense to mean that items following the word are included, without excluding items not specifically mentioned. In addition, reference to an element by the indefinite article ‘a’ or ‘an’ does not exclude the possibility that more than one of the element is present, unless the context clearly requires that there be one and only one of the elements. The indefinite article ‘a’ or ‘an’ thus usually means ‘at least one’.

Hormone-sensitive cancers refer to those types of cancer that grow faster in the presence of particular hormones. This type of cancer is usually treated with hormone therapy. Hormone therapy involves blocking in vivo production or action of these hormones. Therefore, hormone therapy actually is anti-hormone therapy. Examples of hormone sensitive-cancers that occur in female mammals include breast cancer, endometrium cancer and ovarian cancer.

Breast cancer cells can express different types of markers which are used to determine the treatment strategy and give an indication of the clinical outcome. The estrogen receptor (ER) and progesterone receptor (PR) markers are both hormone receptors. ER and PR positive tumor growth is stimulated by estrogens and progesterone. 75% of all breast cancer patients are ER positive. 65% of the ER positive population is also PR positive. The ER/PR positive breast cancers have the most favorable clinical outcome as they are very responsive to anti hormone treatments, e.g. tamoxifen and aromatase inhibitors. If one of these receptors is not expressed by the tumor, hormone therapies are less effective. If none of the two receptors are expressed, the tumor is insensitive to hormone treatments.

Besides the ER and PR, breast cancers can also express the protein HER2/neu. This protein is expressed in approximately 20-25% of all breast cancers. Expression of HER2/neu is correlated with a more aggressive tumor and a poorer clinical outcome compared to HER2/neu negative tumors. Breast cancers expressing HER2/neu are aggressively treated with surgery with adjuvant chemotherapy plus herceptin. Herceptin specifically targets the HER2/neu protein and induces apoptosis in these cells.

If all three markers described above are not expressed the tumor is triple negative. Approximately 15% of all breast cancers are triple negative. Tumor growth does not depend on hormones or on HER2/neu. Therefore, the triple negative tumors are insensitive to conventional anti hormone and herceptin treatments. Currently the only treatment options for this subpopulation are surgery, radiotherapy and aggressive chemotherapy with anthracyclines, taxanes, ixabepilone, or platinum (e.g. cisplatin) agents. However, these approaches usually have a very poor clinical outcome. When these strategies fail, there are no alternatives.

The present invention encompasses the treatment of hormone-sensitive as well as hormone-insensitive cancers. The benefits of the present method of treatment are particularly pronounced when used in the treatment of hormone-insensitive (or hormone independent) cancer.

The present method is particularly suited for treatment of hormone-insensitive cancers that have developed after treatment of hormone sensitive cancers with hormone therapy.

The present method of treatment is advantageously applied to treat a cancer that does not respond to treatment with anti-estrogen, aromatase inhibitor or an inhibitor of 17α hydroxylase/C17,20 lyase (CYP17A1). The present method is particularly suited for treatment of hormone insensitive cancers that have developed after treatment of hormone sensitive cancers with anti-estrogen, aromatase inhibitor or an inhibitor of 17α hydroxylase/C17,20 lyase (CYP17A1).

The present method is particularly suited for treatment of a cancer selected from breast cancer, endometrium cancer and ovarian cancer. The method of the present invention is especially effective in the treatment of breast cancer. According to a particularly preferred embodiment, the present method is employed in the curative or palliative treatment of triple negative breast cancer.

As explained herein before, Fosfestrol in the context of the present invention also encompasses pharmaceutically acceptable salts of Fosfestrol. Pharmaceutically acceptable salts include those formed from cations of alkali metals such as sodium, lithium, potassium, and earth alkali metals such as calcium and magnesium.

In a preferred embodiment the Fosfestrol is an alkali metal salt, notably a sodium and/or a potassium salt. More preferably, the Fosfestrol is in the potassium salt form.

The present method of treatment may be used to treat several kinds of mammals, e.g. humans, horses, cattle etc. The present method is particularly suited for the treatment of humans.

The Fosfestrol dosage may vary depending upon the specific conditions and patients undergoing treatment. The therapeutically effective dosage of the compound can be provided as repeated doses within a prolonged treatment regimen that will yield clinically significant results.

The actual dosage of the compound will vary according to factors such as the disease indication and particular status of the subject such as for example, age, size, fitness, extent of symptoms, susceptibility factors and the like, and other factors such as time and route of administration, other drugs or treatments being administered concurrently. Dosage regimens can be adjusted to provide an optimum therapeutic response.

Typically, the present method comprises administering Fosfestrol in a daily oral amount of at least 700 mg, more preferably of 850-5,000 mg and most preferably of 1,000-1,500 mg.

Expressed differently, it is preferred to administer Fosfestrol orally in a daily amount of at least 6 mg per kg of bodyweight, more preferably of 10-66 mg per kg of bodyweight and most preferably of 13-20 mg per kg of bodyweight.

The duration of the present method of treatment typically exceeds 7 days. More particularly, the present method has a duration of at least 14 days, especially of at least 28 days.

The aforementioned daily amount may be administered once daily of it may be administered in the form of two or more separate doses at more or less regular intervals. According to a particularly preferred embodiment, the present method of treatment comprises orally administering at least two doses per day, more preferably at least two doses of each at least 200 mg Fosfestrol per day, even more preferably it comprises orally administering at least 3 doses of at least 200 mg Fosfestrol per day.

In accordance with a particularly preferred embodiment of the present method the Fosfestrol is orally administered to achieve a plasma level of free DES of at least 1 μg/ml, more preferably of at least 1.5 μg/ml and most preferably of at least 2 μg/ml.

Another aspect of the invention relates to an oral dosage unit comprising at least 500 mg Fosfestrol.

The oral dosage unit of the present invention can advantageously be applied in the curative or palliative treatment of cancer as defined herein before.

The oral dosage units is preferably selected from the group consisting of tablets, granulates, capsules and powders and liquids. Even more preferably, the oral dosage unit is a tablet or a capsule.

The oral dosage units typically have a weight of between 0.5 and 2.0 g, more preferably of 0.7-1.5 g and most preferably of 0.8-1.2 g. In another embodiment, the oral dosage units comprise between 10 and 90 wt. %, more preferably between 20 and 80 wt. % of pharmaceutically acceptable excipient. The pharmaceutically acceptable excipient is suitably selected from coloring agents, flavoring or taste masking agents, diluents, binders, lubricants, disintegrants, stabilizers, surfactants, glidants, plasticizers, preservatives, sweeteners and combinations thereof.

The disintegrants are advantageously chosen from the group consisting of lactose, anhydrous lactose, crospovidone, croscarmellose sodium, sodium starch glycolate, hydroxypropyl cellulose, polacrilin potassium, pregelatinized starch, microcrystalline cellulose and combinations thereof. In a preferred embodiment the oral dosage units comprise up to 7 wt. %, preferably 2-5 wt. % of disintegrants.

The dosage unit of the present invention may suitably take the shape of a compressed tablet. Such a tablet may suitably comprise two or more layers of different composition, for example a core comprising Fosfestrol as defined herein before encased in a coating.

The dosage units of the present inventions are conveniently produced in a tabletting machine. In order to enable easy removal of the tablets from the moulds, the dosage unit typically contains between 0.2 and 4 wt. % of a lubricant or gliding agent. Preferably, the lubricant or gliding agent is selected from the group consisting of talc, sodium stearyl fumarate, magnesium stearate, calcium stearate, hydrogenated castor oil, hydrogenated soybean oil, polyethylene glycol, starches, anhydrous colloidal silica and combinations thereof.

The following examples are meant to further illustrate the invention and some of its preferred embodiments without intending to limit its scope.

### EXAMPLES

### Example 1

The in vitro direct cytotoxicity of DES and Fosfestrol in hormone-sensitive (MCF-7) and hormone-insensitive (MDA-MB231) breast cell lines was tested.

Cells were maintained in vitro in RPMI 1640 containing 10% (v/v) heat inactivated fetal bovine serum (FBS) and 2 mM L-glutamine (growth media) at 37° C. in 5% CO₂ and humidified conditions. Cells were harvested, washed, re-suspended into growth medium and counted. The cells were re-suspended into assay media (RPMI 1640+1% (v/v) heat inactivated FBS+ and 2 mM L-glutamine) at 0.5-1×10⁵ cells/ml (dependent on cell type), and plated into 96-well assay plates (Corning, black-wall plates) and 50 μl/well aliquots.

Plates were incubated O/N at 37° C. in 5% humidified CO₂ prior to addition of the compounds. DES was dissolved in 100% DMSO at stock concentration of 60 mM. Fosfestrol was dissolved in sterile water at stock concentration of 60 mM.

Stocks of all compounds were then serially diluted and final concentration to which cells were exposed were: 300, 150, 75, 37.5, 18.75, 9.4, 4.7, 2.3, 1.2 and 0.6 μM. Positive control was Cisplatin. Cisplatin was diluted in 1% FBS medium to give a stock concentration of 1 mM. Cisplatin stock was serial diluted and final concentration to which cells were exposed was: 100, 50, 25, 12.5, 6.3, 31, 1.6, 0.8, 0.4, 0.2 μM.

Plates were incubated for 72 hrs at 37° C. in 5% humidified CO-2 after addition of the compounds. Viability of the cells was assessed with the Cell titer blue® (Promega) assay. 10 μl of Cell titer Blue™ reagents was added to each test/blank well. Plates were incubated for 3 hrs at 37° C. in 5% humidified CO₂ prior to analysis. Fluorescence was measured with a Flex II station plate reader. Excitation wavelength was 570 nm, emission wave length was 600 nm, cut off was 590 nm. Raw data was processed by GraphPad Prism to calculate mean, standard deviation and IC₅₀ values.

The results so obtained are shown in Table 1.

TABLE 1

IC₅₀ value (μM)

| | MCF7 Cells | MDA-MB-231 Cells |
|---|---|---|
| DES | 3.9 | 6.7 |
| Fosfestrol | 168 | 285 |
| Cisplatin (control) | 2.8 | 13 |

### Conclusion

These results show that DES and Fosfestrol are both cytotoxic in vitro in MCF7 and MDA-MB231 breast cancer cell lines. Of the two compounds, DES is the most cytotoxic. This finding is in sharp contrast to the articles by Dr Schneider that showed (a) that Fosfestrol was more cytotoxic than DES and (b) that DES was not cytotoxic in hormone independent mammary tumors.

### Example 2

A phase I patient study is conducted in advanced breast cancer patients to explore the side effect profile of oral Fosfestrol treatment in these patients.

All patients included undergone at least one prior treatment.

Patients are treated with three times 250 mg/d oral Fosfestrol for 4 weeks. Total Fosfestrol dose per day is 750 mg. All other treatments are stopped during this study.

Treatment is accompanied by minor toxicities and no thromboembolic side effects are detected.

### Conclusion

This study shows that Fosfestrol in safe to use in breast cancer patients.

### Example 3

A 1 kg batch of 500 mg Fosfestrol tablets was prepared by direct compression. The API and excipients were passed over a 0.85 mm sieve. 500 gram of Fosfestrol tetrasodium was blended with 435 gram of Silicified Microcrystalline Cellulose

(Prosolv smcc 90™) and 50 gram of croscarmellose sodium (Ac-di-Sol™) for 20 minutes in a V-blender. Added to the mixture was 15 grams of magnesium stearate and blending was continued for 5 minutes. Tablets of 1,000 mg each were prepared on a Korsch EKO, using caplet shaped punches.

1-17. (canceled)

18. A method of curative or palliative treatment of cancer in female mammals, comprising orally administering to a mammal in need thereof a daily amount of at least 500 mg fosfestrol (diethylstilbestrol diphosphate).

19. The method according to claim 18, wherein the cancer is a hormone-insensitive cancer.

20. The method according to claim 19, wherein the hormone-insensitive cancer has developed after treatment of a hormone sensitive cancer with anti-estrogen, aromatase inhibitor or an inhibitor of 17α hydroxylase/C17,20 lyase (CYP17A1).

21. The method according to claim 20, wherein the hormone-insensitive cancer has developed after treatment of a hormone sensitive cancer with an inhibitor of 17α hydroxylase/C17,20 lyase (CYP17A1).

22. The method according to claim 21, wherein the hormone-insensitive cancer has developed after treatment of a hormone sensitive cancer with Abiraterone.

23. The method according to claim 18, wherein the cancer is selected from the group consisting of breast cancer, endometrium cancer and ovarian cancer.

24. The method according to claim 22, wherein the cancer is breast cancer.

25. The method according to claim 23, wherein the breast cancer is triple negative breast cancer.

26. The method according to claim 18, wherein the method comprises orally administering a daily amount of 500-5,000 mg fosfestrol.

27. The method according to claim 18, wherein the fosfestrol is administered daily for at least 7 days.

28. The method according to claim 18, wherein the method comprises orally administering at least 6 mg of fosfestrol per kg of bodyweight.

29. The method according to claim 18, wherein a plasma level of free DES above 1 microgram/m is achieved.

30. The method according to claim 18, wherein the method comprises at least twice daily oral administration of fosfestrol.

31. The method according to claim 18, wherein the mammal is a human.

32. An oral dosage unit selected from a tablet or a capsule, the oral dosage unit comprising at least 500 mg of Fosfestrol.

33. The oral dosage unit according to claim 31, wherein the oral dosage unit has a weight of 0.5-2.0 g.

34. The oral dosage unit according to claim 31, comprising 10-90 wt. % of one or more pharmaceutically acceptable excipients.
